(19) United States
(12) Patent Application Publication
(10) Pub. No.: US 2023/0263501 A1
(43) Pub. Date: Aug. 24, 2023

(54) DETERMINING HEART RATE BASED ON A SEQUENCE OF ULTRASOUND IMAGES

(71) Applicant: EchoNous, Inc., Redmond, WA (US)

(72) Inventor: Fan Zhang, Bellevue, WA (US)

(21) Appl. No.: 17/678,813

(22) Filed: Feb. 23, 2022

(51) Int. Cl. A61B 8/02; A61B 8/08; A61B 8/00; G06N 3/04

(52) U.S. Cl. CPC A61B 8/02; A61B 8/488; A61B 8/5207; A61B 8/461; A61B 8/5223; G06N 3/0472

(57) ABSTRACT

A facility for determining a heart rate of a person is described. The facility receives ultrasound data collected from the person at each of a number of times during a period of time, such as a sequence of B-mode images, or an M-mode image. For each of these times, the facility compresses the ultrasound date relating to the time to obtain a single-value representation of that ultrasound data; adds the obtained single-value representation to a time-ordered buffer of single-value representation of ultrasound data from earlier times; and processes the buffer to determine a heart rate of the person, such as by performing procedural peak-finding or applying a machine learning model to predict heart rate.

### BACKGROUND

Ultrasound imaging is a useful medical imaging modality. For example, internal structures of a patient's body may be imaged before, during or after a therapeutic intervention. Also, qualitative and quantitative observations in an ultrasound image can be a basis for diagnosis. For example, ventricular volume determined via ultrasound is a basis for diagnosing, for example, ventricular systolic dysfunction and diastolic heart failure.

A healthcare professional typically holds a portable ultrasound probe, sometimes called a “transducer,” in proximity to the patient and moves the transducer as appropriate to visualize one or more target structures in a region of interest in the patient. A transducer may be placed on the surface of the body or, in some procedures, a transducer is inserted inside the patient's body. The healthcare professional coordinates the movement of the transducer so as to obtain a desired representation on a screen, such as a two-dimensional cross-section of a three-dimensional volume.

Many ultrasound systems perform imaging in multiple imaging modes; two of these are B-mode and M-mode. B-mode is the system's default imaging mode, in which the system displays echoes in two dimensions by assigning a brightness level based on the echo signal amplitude. M-mode, also known as Motion Mode, provides a trace of the image displayed over time. A single beam of ultrasound is transmitted, and reflected signals are displayed as dots of varying intensities, which create lines across the screen.

Particular views of an organ or other tissue or body feature (such as fluids, bones, joints or the like) can be clinically significant. Such views may be prescribed by clinical standards as views that should be captured by the ultrasound operator, depending on the target organ, diagnostic purpose or the like.

In some ultrasound images, it is useful to identify anatomical structures visualized in the image. For example in an ultrasound image view showing a particular organ, it can be useful to identify constituent structures within the organ. As one example, in some views of the heart, constituent structures are visible, such as the left and right atria; left and right ventricles; and aortic, mitral, pulmonary, and tricuspid valves.

Existing software solutions have sought to identify such structures automatically. These existing solutions seek to “detect” a structure by specifying a horizontal bounding box in which the structure is visible, or “segment” the structure by identifying the individual pixels in the image that show the structure.

“Heart rate” is a medical metric indicating the rate at which the heart fills with and pumps out blood, often expressed in the unit “beats per minute,” or “bpm.” The heart rate metric is commonly measured using a stethoscope or electrocardiogram.

### BRIEF DESCRIPTION OF THE DRAWINGS

FIG. 1 is a block diagram showing some of the components typically incorporated in at least some of the computer systems and other devices on which the facility operates.

FIG. 2 is a block diagram showing some of the components typically incorporated in at least some of the computer systems and other devices on which the facility operates.

FIG. 3 is a data flow diagram showing the facility's operation at a high level.

FIG. 4 is a model architecture diagram showing a model architecture used by facility in some embodiments to accommodate a grid of rectangle reference regions.

FIG. 5 is a data flow diagram showing the facility's implementation of the rhythm estimator in some embodiments.

FIG. 6 is a flowchart showing a process performed by the facility in some embodiments in order to perform peak finding in the time-series array of single-value representations of the input(s).

FIG. 7 is a data flow diagram showing the facility's generation of a latent representation of each time slice of an M-mode ultrasound image as a basis for populating the buffer used by the facility to determine heart rate.

### DETAILED DESCRIPTION

The inventor has recognized that ultrasound is commonly used in emergency medicine settings where healthcare providers examine a patient's health condition on the spot, perform quick diagnosis, and execute optimal procedure. In this setting, heart rate can provide useful information on a patient's cardiac functionality. There is seldom time or space to measure heart rate by conventional means dedicated to this purpose, however.

It has occurred to the inventor that automatically determining heart rate based upon a sequence of ultrasound images captured for a different primary purpose can provide the benefit of an accurate heart rate measurement without the need to have on hand special-purpose instruments such as stethoscope or electrocardiogram, or to devote the space or provider time needed to operate them.

In response, the inventor has conceived and reduced to practice a software and/or hardware facility that automatically determines heart rate from a sequence of ultrasound images (“the facility”). In various embodiments, the facility determines heart rate using data produced in the B-mode and/or M-mode imaging modes.

In some embodiments, the facility performs peak finding, period calculation, and post-processing on a time-series signal. In some embodiments, the facility generates this time-series signal by a process of filtering, pooling, and buffering an ultrasound-related input. In various embodiments, this input is any combination of a series of raw ultrasound images; a series of vectors of view logits indicating the likelihood that each ultrasound image was captured from each of a number of possible views; and/or a series of object detection result that identifies, in each ultrasound image, objects of different types detected at particular locations. In some embodiments, these ultrasound images are captured in B-mode. In some embodiments, the facility produces the view logit vectors and/or the object detection results using a machine learning model, such as a convolutional neural network or other artificial neural network.

In some embodiments, the facility applies an auto-encoder model made using multi-layer perceptrons (MLP) to reconstruct the vertical lines in an M-mode ultrasound image captured in the M-mode imaging mode. In particular, in the auto-encoder model constructed by the facility, a first MLP reduces, or “encodes,” the signal of the M-mode image to a small latent representation of the signal. A second auto-encoder expands, or “decodes,” the latent representation to a reconstructed version of the signal. After training the model, the facility operates the trained first multi-layer perceptron of the auto-encoder model to reduce the signal of an M-mode image to a latent representation, then applies the process of filtering, pooling, buffering, peak-finding, period calculation, and post-processing described above. In some embodiments, the facility augments the sequence of ultrasound images for display to include visual information about the patient's determined heart rate, such as a digital display of heart rate, a bar graph or analog gauge showing heart rate, a heart rate vs. time graph, etc.

By performing in some or all of these ways, the facility determines a patient's heart rate from a sequence of the patient's ultrasound images, without the need to possess or operate any separate instruments dedicated to that task. Also, the facility incorporates information about the patient's determined heart rate into display of the patient's sequence of ultrasound images so that both can easily be viewed together by the ultrasound operator or other healthcare providers.

Additionally, the facility improves the functioning of computer or other hardware, such as by reducing the dynamic display area, processing, storage, and/or data transmission resources needed to perform a certain task, thereby enabling the task to be permitted by less capable, capacious, and/or expensive hardware devices, and/or be performed with lesser latency, and/or preserving more of the conserved resources for use in performing other tasks. For example, the facility in many cases renders unnecessary a stethoscope and/or an electrocardiogram machine, so that high-quality emergency medical care can be provided without the expense or effort of acquiring and maintaining these instruments.

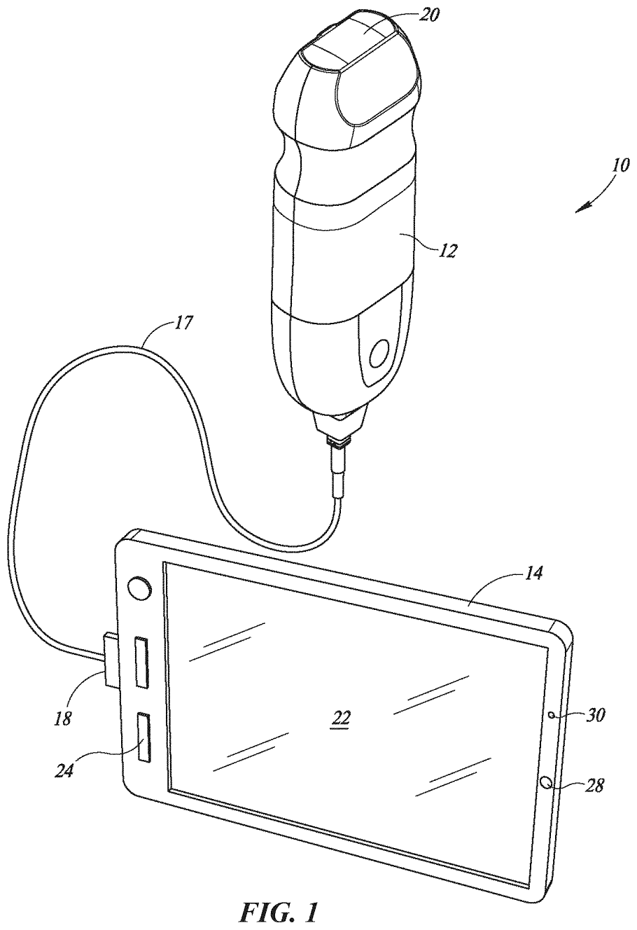

FIG. 1 is a schematic illustration of a physiological sensing device, in accordance with one or more embodiments of the present disclosure. The device 10 includes a probe 12 that, in the illustrated embodiment, is electrically coupled to a handheld computing device 14 by a cable 17. The cable 17 includes a connector 18 that detachably connects the probe 12 to the computing device 14. The handheld computing device 14 may be any portable computing device having a display, such as a tablet computer, a smartphone, or the like. In some embodiments, the probe 12 need not be electrically coupled to the handheld computing device 14, but may operate independently of the handheld computing device 14, and the probe 12 may communicate with the handheld computing device 14 via a wireless communication channel.

The probe 12 is configured to transmit an ultrasound signal toward a target structure and to receive echo signals returning from the target structure in response to transmission of the ultrasound signal. The probe 12 includes an ultrasound sensor 20 that, in various embodiments, may include an array of transducer elements (e.g., a transducer array) capable of transmitting an ultrasound signal and receiving subsequent echo signals.

The device 10 further includes processing circuitry and driving circuitry. In part, the processing circuitry controls the transmission of the ultrasound signal from the ultrasound sensor 20. The driving circuitry is operatively coupled to the ultrasound sensor 20 for driving the transmission of the ultrasound signal, e.g., in response to a control signal received from the processing circuitry. The driving circuitry and processor circuitry may be included in one or both of the probe 12 and the handheld computing device 14. The device 10 also includes a power supply that provides power to the driving circuitry for transmission of the ultrasound signal, for example, in a pulsed wave or a continuous wave mode of operation.

The ultrasound sensor 20 of the probe 12 may include one or more transmit transducer elements that transmit the ultrasound signal and one or more receive transducer elements that receive echo signals returning from a target structure in response to transmission of the ultrasound signal. In some embodiments, some or all of the transducer elements of the ultrasound sensor 20 may act as transmit transducer elements during a first period of time and as receive transducer elements during a second period of time that is different than the first period of time (i.e., the same transducer elements may be usable to transmit the ultrasound signal and to receive echo signals at different times).

The computing device 14 shown in FIG. 1 includes a display screen 22 and a user interface 24. The display screen 22 may be a display incorporating any type of display technology including, but not limited to, LCD or LED display technology. The display screen 22 is used to display one or more images generated from echo data obtained from the echo signals received in response to transmission of an ultrasound signal, and in some embodiments, the display screen 22 may be used to display color flow image information, for example, as may be provided in a Color Doppler imaging (CDI) mode. Moreover, in some embodiments, the display screen 22 may be used to display audio waveforms, such as waveforms representative of an acquired or conditioned auscultation signal.

In some embodiments, the display screen 22 may be a touch screen capable of receiving input from an operator that touches the screen. In such embodiments, the user interface 24 may include a portion or the entire display screen 22, which is capable of receiving operator input via touch. In some embodiments, the user interface 24 may include one or more buttons, knobs, switches, and the like, capable of receiving input from an operator of the ultrasound device 10. In some embodiments, the user interface 24 may include a microphone 30 capable of receiving audible input, such as voice commands.

The computing device 14 may further include one or more audio speakers 28 that may be used to output acquired or conditioned auscultation signals, or audible representations of echo signals, blood flow during Doppler ultrasound imaging, or other features derived from operation of the device 10.

The probe 12 includes a housing, which forms an external portion of the probe 12. The housing includes a sensor portion located near a distal end of the housing, and a handle portion located between a proximal end and the distal end of the housing. The handle portion is proximally located with respect to the sensor portion.

The handle portion is a portion of the housing that is gripped by an operator to hold, control, and manipulate the probe 12 during use. The handle portion may include gripping features, such as one or more detents, and in some embodiments, the handle portion may have a same general shape as portions of the housing that are distal to, or proximal to, the handle portion.

The housing surrounds internal electronic components and/or circuitry of the probe 12, including, for example, electronics such as driving circuitry, processing circuitry, oscillators, beamforming circuitry, filtering circuitry, and the like. The housing may be formed to surround or at least partially surround externally located portions of the probe 12, such as a sensing surface. The housing may be a sealed housing, such that moisture, liquid or other fluids are prevented from entering the housing. The housing may be formed of any suitable materials, and in some embodiments, the housing is formed of a plastic material. The housing may be formed of a single piece (e.g., a single material that is molded surrounding the internal components) or may be formed of two or more pieces (e.g., upper and lower halves) which are bonded or otherwise attached to one another.

In some embodiments, the probe 12 includes a motion sensor. The motion sensor is operable to sense a motion of the probe 12. The motion sensor is included in or on the probe 12 and may include, for example, one or more accelerometers, magnetometers, or gyroscopes for sensing motion of the probe 12. For example, the motion sensor may be or include any of a piezoelectric, piezoresistive, or capacitive accelerometer capable of sensing motion of the probe 12. In some embodiments, the motion sensor is a tri-axial motion sensor capable of sensing motion about any of three axes. In some embodiments, more than one motion sensor 16 is included in or on the probe 12. In some embodiments, the motion sensor includes at least one accelerometer and at least one gyroscope.

The motion sensor may be housed at least partially within the housing of the probe 12. In some embodiments, the motion sensor is positioned at or near the sensing surface of the probe 12. In some embodiments, the sensing surface is a surface which is operably brought into contact with a patient during an examination, such as for ultrasound imaging or auscultation sensing. The ultrasound sensor 20 and one or more auscultation sensors are positioned on, at, or near the sensing surface.

In some embodiments, the transducer array of the ultrasound sensor 20 is a one-dimensional (1D) array or a two-dimensional (2D) array of transducer elements. The transducer array may include piezoelectric ceramics, such as lead zirconate titanate (PZT), or may be based on microelectromechanical systems (MEMS). For example, in various embodiments, the ultrasound sensor 20 may include piezoelectric micromachined ultrasonic transducers (PMUT), which are microelectromechanical systems (MEMS)-based piezoelectric ultrasonic transducers, or the ultrasound sensor 20 may include capacitive micromachined ultrasound transducers (CMUT) in which the energy transduction is provided due to a change in capacitance.

The ultrasound sensor 20 may further include an ultrasound focusing lens, which may be positioned over the transducer array, and which may form a part of the sensing surface. The focusing lens may be any lens operable to focus a transmitted ultrasound beam from the transducer array toward a patient and/or to focus a reflected ultrasound beam from the patient to the transducer array. The ultrasound focusing lens may have a curved surface shape in some embodiments. The ultrasound focusing lens may have different shapes, depending on a desired application, e.g., a desired operating frequency, or the like. The ultrasound focusing lens may be formed of any suitable material, and in some embodiments, the ultrasound focusing lens is formed of a room-temperature-vulcanizing (RTV) rubber material.

In some embodiments, first and second membranes are positioned adjacent to opposite sides of the ultrasound sensor 20 and form a part of the sensing surface. The membranes may be formed of any suitable material, and in some embodiments, the membranes are formed of a room-temperature-vulcanizing (RTV) rubber material. In some embodiments, the membranes are formed of a same material as the ultrasound focusing lens.

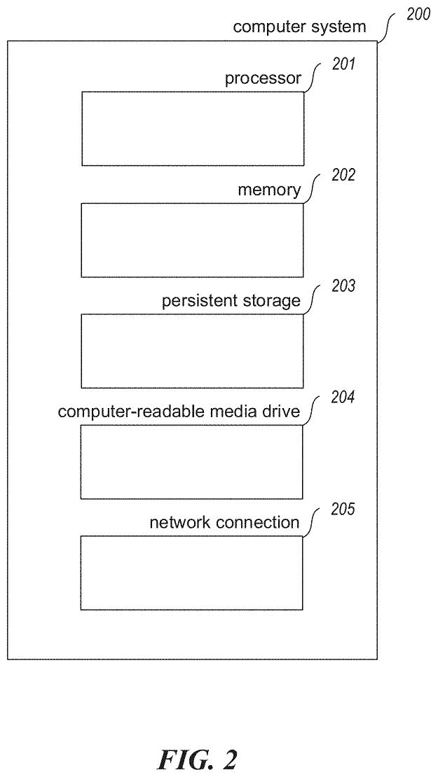

FIG. 2 is a block diagram showing some of the components typically incorporated in at least some of the computer systems and other devices on which the facility operates. In various embodiments, these computer systems and other devices 200 can include server computer systems, cloud computing platforms or virtual machines in other configurations, desktop computer systems, laptop computer systems, netbooks, mobile phones, personal digital assistants, televisions, cameras, automobile computers, electronic media players, physiological sensing devices, and/or their associated display devices, etc. In various embodiments, the computer systems and devices include zero or more of each of the following: a processor 201 for executing computer programs and/or training or applying machine learning models, such as a CPU, GPU, TPU, NNP, FPGA, or ASIC; a computer memory 202 for storing programs and data while they are being used, including the facility and associated data, an operating system including a kernel, and device drivers; a persistent storage device 203, such as a hard drive or flash drive for persistently storing programs and data; a computer-readable media drive 204, such as a floppy, CD-ROM, or DVD drive, for reading programs and data stored on a computer-readable medium; and a network connection 205 for connecting the computer system to other computer systems to send and/or receive data, such as via the Internet or another network and its networking hardware, such as switches, routers, repeaters, electrical cables and optical fibers, light emitters and receivers, radio transmitters and receivers, and the like. While computer systems configured as described above are typically used to support the operation of the facility, those skilled in the art will appreciate that the facility may be implemented using devices of various types and configurations, and having various components.

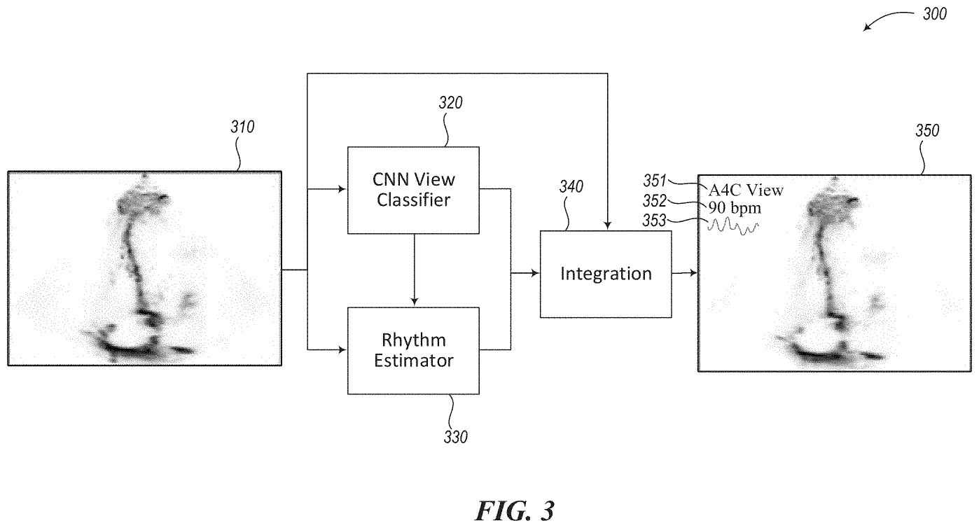

FIG. 3 is a data flow diagram showing the facility's operation at a high level. The diagram 300 shows the facility's augmentation of a raw ultrasound image 310—such as a B-mode ultrasound image, or an M-mode ultrasound image—with additional visual information: a prediction 351 of the view from which the ultrasound image was captured, a digital display 352 of a heart rate determined for the patient by the facility, and a predicted heart contraction trace 353, all shown in an augmented copy 350 of the ultrasound image that can be displayed to the ultrasound operator and/or others. In particular, the data flow diagram shows that the raw ultrasound image is supplied both to a CNN view classifier 320 and a rhythm estimator 330. The CNN view classifier is a convolutional neural network whose architectural details are described below. In various embodiments, the CNN view classifier—or other machine learning models of various types—make predictions from the ultrasound image such as the view from which the ultrasound image was captured; object identification results for the ultrasound image identifying object types in the ultrasound image and their locations therein; or both. In some embodiments, some or all of the predictions made by the view classifier from the ultrasound image are supplied to the rhythm estimator. In some embodiments, some or all of these predictions are supplied to an integration module 340 for augmenting the ultrasound image. The rhythm estimator uses the ultrasound image, as well as in some cases view classification results and/or object identification results, to estimate the cardiac rhythm of the patient, and subsequently, the patient's heart rate. The integration module 340 receives the raw image, and augments it with information determined from the raw image by the facility, including, for example, heart rate information, view identification, object identification, etc. As a result of this action by the integration module, the facility produces augmented ultrasound image 350 for display.

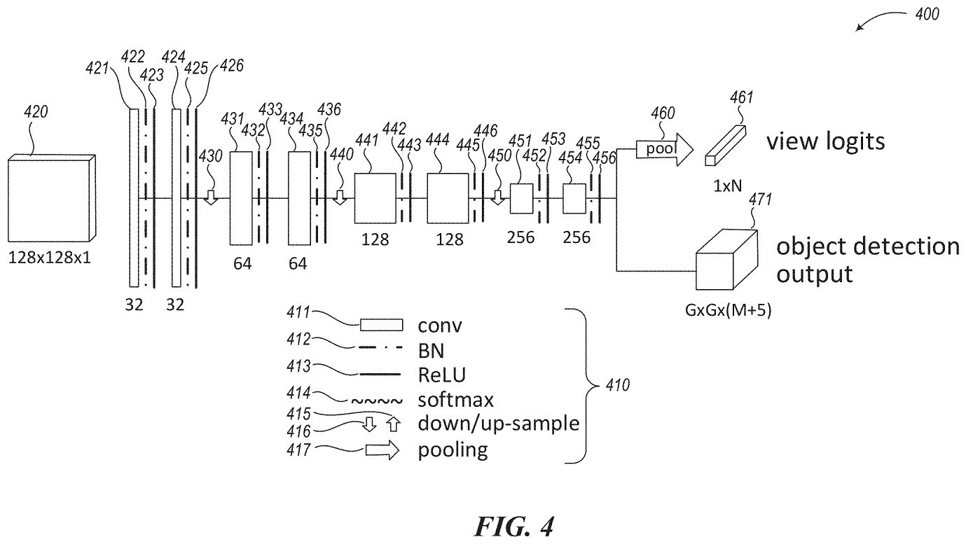

FIG. 4 is a model architecture diagram showing a model architecture used by facility in some embodiments to accommodate a grid of rectangle reference regions, such as rectangle reference regions in a B-mode image. The model 400 is shown with respect to a key 410. The key shows symbols used in the diagram to represent 2D Convolutional layers 411, 2D Batch normalization layer 412, Leaky ReLU activation function layers 413, softmax layers 414, down-sample layers 615, up-sample layers 416, and pooling layers 417.

The model takes a 128×128×1 ultrasound image 420 as its input, and produces two outputs: a vector 461 of N view logits, each value indicating the probability that the ultrasound image was captured from a different standard ultrasound view; an object detection output 471, which in some embodiments is a 3-dimensional array where two of the dimensions identify different positions in the ultrasound image, and the third dimension identifies the type, size, and location of objects identified near those positions. The model first subjects the input ultrasound image to a convolutional block made up of 2D convolutional layer 421, 2D batch normalization layer 422, and leaky relu activation function layer 423. The model then proceeds to a convolutional block made up of 2D convolutional layer 424, 2D batch normalization layer 425, and leaky relu activation function layer 426. The model then proceeds to a downsample layer 430. The model then proceeds to a convolutional block made up of 2D convolutional layer 431, 2D batch normalization layer 432, and leaky relu activation function layer 433. The model then proceeds to a convolutional block made up of 2D convolutional layer 434, 2D batch normalization layer 435, and leaky relu activation function layer 436. The model then proceeds to a downsample layer 440. The model then proceeds to a convolutional block made up of 2D convolutional layer 441, 2D batch normalization layer 442, and leaky relu activation function layer 443. The model then proceeds to a convolutional block made up of 2D convolutional layer 444, 2D batch normalization layer 445, and leaky relu activation function layer 446. The model then proceeds to a downsample layer 450. The model then proceeds to a convolutional block made up of 2D convolutional layer 451, 2D batch normalization layer 452, and leaky relu activation function layer 453. The model then proceeds to a convolutional block made up of 2D convolutional layer 454, 2D batch normalization layer 455, and leaky relu activation function layer 453. The model then branches to produce the view prediction 461 via a pooling layer 460, and the object detention output 471. In various embodiments, the facility uses a variety of neural network architectures and other machine learning model architectures to produce similar results. In some embodiments, the network produces one or the other of the shown outputs, but not both.

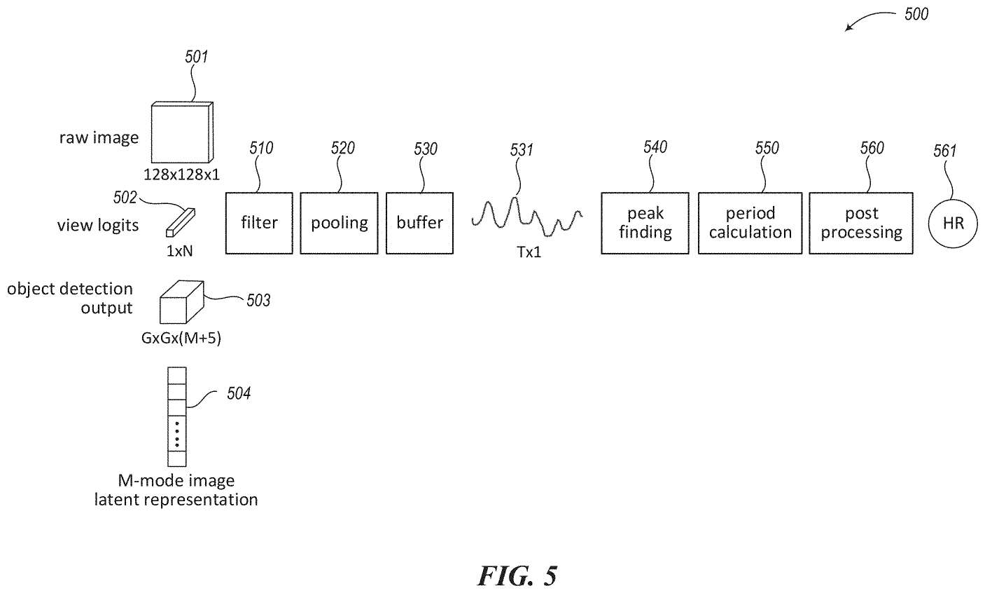

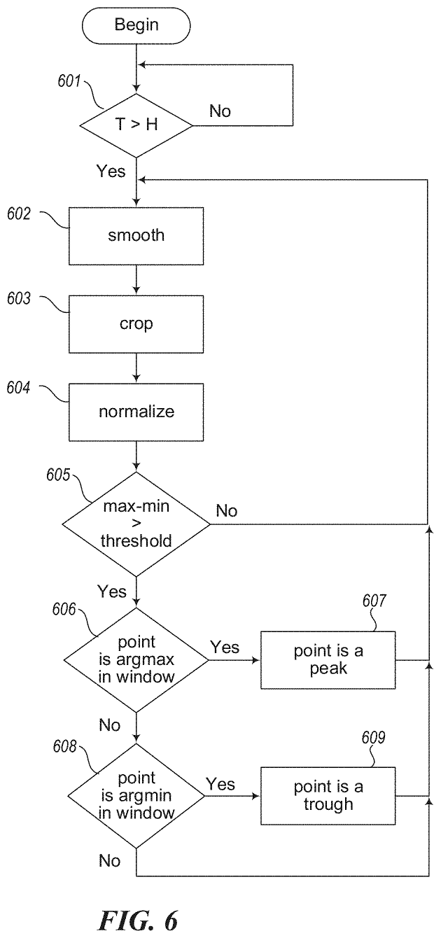

FIG. 5 is a data flow diagram showing the facility's implementation of the rhythm estimator in some embodiments. The diagram 500 shows that the facility uses one or more of the following inputs: the raw ultrasound image 501, the view prediction results 502 or results of other image classification processes, the object detection results 503, the latent representation created by the encoding stage of an auto-encoder model, and/or image segmentation results (not shown). In various embodiments, the facility uses various combinations of one or more of these inputs to determine a heart rate 561. In particular, in some embodiments, the facility performs a filtering step 510 against the used input(s), in some embodiments using such filtering techniques as a canny edge detector, or a Laplacian or Gaussian filter. In some embodiments, the facility then performs a pooling step 520 to aggregate the post-filtering input information into a single value, in some embodiments using an aggregation function such as maximum, sum, mean, or median. In some embodiments, the facility then performs a buffering step 530 to accumulate these single-value representations of inputs representing already-received raw images into a time-series array whose length is limited to a number of entries specified by the variable T. Sample contents 531 of the buffer constructed by the buffering step are shown. In some embodiments, the facility then performs a peak finding step 540 against the time-series array stored in the buffer. Additional details about the peak finding step are discussed below in connection with FIG. 6. In some embodiments, the facility then performs a period calculation step 550 against the peaks identified in the peak finding step. In some embodiments, this involves identifying the distance and time between each successive pair of identified peaks, and aggregating these using an aggregation function such as mean or median. In some embodiments, the facility next performs a post-processing step 560 to filter or smooth the calculated period, and invert it to obtain heart rate.

FIG. 6 is a flowchart showing a process performed by the facility in some embodiments in order to perform peak finding in the time-series array of single-value representations of the input(s). In act 601, the facility accumulates a time-series array of adequate length by, until the number of entries in the array is equal to the maximum size of the array, remaining in act 601. In act 602, the facility smooths the time-series array, in some embodiments using such techniques as Gaussian filtering, or averaging. In act 603, the facility crops the head and tail of the time interval removing the first few and the last few entries. In act 604, the facility normalizes the time-series, such as by projecting its values on to a range between 0 and 1. In act 605, if the maximum remaining value in the time series is more than a threshold amount above the minimum value in the time series, then the facility continues in act 606, else the facility determines that the present value is not a valid peak or a trough, and continues in act 602 process the next version of the time series in which the single-value representation of the next ultrasound image is added to the time-series, and the oldest value is removed from the time series. In act 606, if the most recent point is a maximum in the time series, then the facility continues in act 607, else the facility continues in act 608. In act 607, the facility determines that the most recent value in the time series is a peak. After act 607, the facility continues in act 602 to process the next version of the time series. In act 608, if the present value of the time series is a minimum in the time series, then the facility continues in act 609, else the facility continues in act 602 without identifying the present value as a peak or a trough to process the next version of the time series. In act 609, the facility determines that the current value of the time series is a trough. After act 609, the facility continues in act 602 to process the next version of the time series.

Those skilled in the art will appreciate that the acts shown in FIG. 6 may be altered in a variety of ways. For example, the order of the acts may be rearranged; some acts may be performed in parallel; shown acts may be omitted, or other acts may be included; a shown act may be divided into subacts, or multiple shown acts may be combined into a single act, etc.

In some embodiments (not shown), rather than performing peak-finding and calculating periods between peaks, the facility subjects the contents of the time-series array to a machine learning model trained to predict heart rate from time-series array contents. In some embodiments, the facility uses a direct regression machine learning model for this purpose. In some embodiments, the facility trains the machine learning model for predicting heart rate, such as by performing the described process on a number of experimental human subjects, and contemporaneously determining their heart rate by another means, such as by using a stethoscope and/or electrocardiogram. The facility trains machine the learning model with these observations to predict heart rate as the dependent variable from time-series array contents as the independent variable.

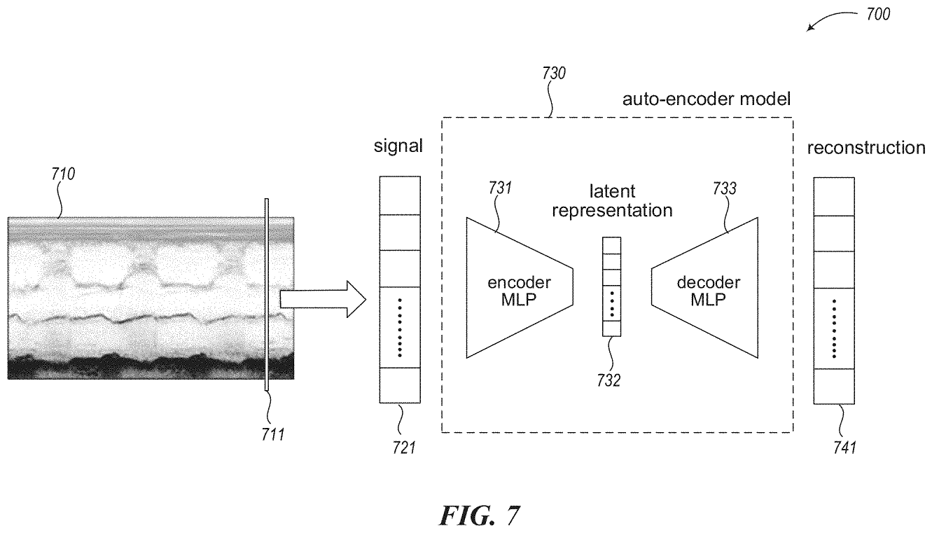

FIG. 7 is a data flow diagram showing the facility's generation of a latent representation of each time slice of an M-mode ultrasound image as a basis for populating the buffer used by the facility to determine heart rate. The diagram 700 shows an M-mode ultrasound image 710 in which each vertical line constitutes a signal captured at a different point in time. For example, vertical line 711 shows a signal for a time near the end of the range of times represented in the M-mode image. The facility applies to each of these vertical lines—shown as signal vector 721—an auto-encoder model 730. This model is made up of a first, encoder multi-layer perceptron 731 (or other encoding stage) that encodes the signal into a much smaller latent representation 732 of the signal, as well as a second, decoder multi-layer perceptron 733 (or other decoding stage) that decodes that latent representation to obtain a reconstruction 741 of the signal. After the auto-encoder model is trained in this way, the facility uses the encoder multi-layer perceptron to generate a latent representation of each vertical line of the subject patient's M-mode image, which is used by the facility as an input to the processing pipeline 500 shown in FIG. 5.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A system, comprising:
an ultrasound transducer; and
a computing device, the computing device comprising:
a communication interface configured to directly receive ultrasound echo data sensed by the ultrasound transducer from a person, the received ultrasound echo data comprising a sequence of ultrasound images; and
a processor configured to:
for each ultrasound image of at least a portion of the ultrasound images of the sequence, in response to receipt of the ultrasound image by the communication interface:
access a multivalued representation of the ultrasound image;
pool values of the multivalued representation of the ultrasound image to obtain a single-value representation of the ultrasound image; and
add the obtained single-value representation of the ultrasound image to a time-ordered buffer window of single-value representations of ultrasound images of the sequence from earlier times.

2. The system of claim 1, the processor further configured to:
for each ultrasound image of the at least a portion of the ultrasound images of the sequence:
determine whether the single-value representation of the ultrasound image is a peak within the buffer window;
among two or more single-value representations of ultrasound images of the sequence determined to be peaks, determine an average time period between successive pairs of these single-value representations of ultrasound images of the sequence determined to be peaks; and
invert the determined average time period to obtain a heart rate.

3. The system of claim 1, the processor further configured to:
apply a trained machine learning model to the contents of the buffer window to predict a heart rate.

4. The system of claim 3 wherein the machine learning model is a direct regression model.

5. The system of claim 3, the processor further configured to train the machine learning model using a plurality of observations each comprising buffer window contents for a human subject and a heart rate independently and contemporaneously determined for the human subject.

6. The system of claim 1, wherein each image in the received sequence of ultrasounds images is a B-mode ultrasound image.

7. A method in a computing system, comprising:
receiving a sequence of ultrasound images of a person;
for each ultrasound image of at least a portion of the ultrasound images of the sequence:
accessing a multivalued representation of the ultrasound image;
pooling values of the multivalued representation of the ultrasound image to obtain a single-value representation of the ultrasound image; and
adding the obtained single-value representation of the ultrasound image to a time-ordered buffer window of single-value representations of ultrasound images of the sequence from earlier times.

8. The method of claim 7, further comprising:
for each ultrasound image of the at least a portion of the ultrasound images of the sequence:
determining whether the single-value representation of the ultrasound image is a peak within the buffer window;
among two or more single-value representations of ultrasound images of the sequence determined to be peaks, determining an average time period between successive pairs of these single-value representations of ultrasound images of the sequence determined to be peaks; and
inverting the determined average time period to obtain a heart rate.

9. The method of claim 7, further comprising applying a trained machine learning model to the contents of the buffer window to predict a heart rate.

10. The method of claim 9 wherein the machine learning model is a direct regression model.

11. The method of claim 9, further comprising training the machine learning model using a plurality of observations each comprising buffer window contents for a human subject and a heart rate independently and contemporaneously determined for the human subject.

12. The method of claim 7, further comprising causing the obtained heart rate to be displayed.

13. The method of claim 7, further comprising causing the obtained heart rate to be stored in connection with identifying information for the person.

14. The method of claim 7, further comprising:
for each ultrasound image of the at least a portion of the ultrasound images of the sequence:
performing filtering on the multivalued representation of the ultrasound image before the multivalued representation of the ultrasound image is accessed.

15. The method of claim 7 wherein the multivalued representation of the ultrasound image is the ultrasound image itself.

16. The method of claim 7 wherein the multivalued representation of the ultrasound image is a set of object detection results obtained for the ultrasound image.

17. The method of claim 16, further comprising:
for each ultrasound image of the at least a portion of the ultrasound images of the sequence:
applying a trained machine learning model to the ultrasound image to produce the set of object detection results.

18. The method of claim 17, further comprising:
using ultrasound images to train the applied machine learning model.

19. The method of claim 7 wherein the multivalued representation of the ultrasound image is a set of segmentation results obtained for the ultrasound image.

20. The method of claim 7 wherein the multivalued representation of the ultrasound image is a set of image classification results obtained for the ultrasound image.

21. The method of claim 7 wherein the multivalued representation of the ultrasound image is a vector of values, each value of the vector corresponding to a different ultrasound view and representing a determined probability that the ultrasound image was captured from that ultrasound view.

22. The method of claim 21, further comprising:
for each ultrasound image of the at least a portion of the ultrasound images of the sequence:
applying a trained machine learning model to the ultrasound image to produce the vector of values.

23. The method of claim 22, further comprising:
using ultrasound images to train the applied machine learning model.

24. One or more computer memory units collectively having contents configured to cause a computing system to perform a method, the method comprising:
receiving a sequence of ultrasound images of a person;
for each ultrasound image of at least a portion of the ultrasound images of the sequence:
accessing a multivalued representation of the ultrasound image;
pooling values of the multivalued representation of the ultrasound image to obtain a single-value representation of the ultrasound image; and
adding the obtained single-value representation of the ultrasound image to a time-ordered buffer window of single-value representation of ultrasound images of the sequence from earlier times.

25. The one or more computer memory units of claim 24, the method further comprising:
for each ultrasound image of the at least a portion of the ultrasound images of the sequence:
determining whether the single-value representation of the ultrasound image is a peak within the buffer window;
among two or more single-value representations of ultrasound images of the sequence determined to be peaks, determining an average time period between successive pairs of these single-value representations of ultrasound images of the sequence determined to be peaks; and
inverting the determined average time period to obtain a heart rate.

26. The one or more computer memory units of claim 24, the method further comprising applying a trained machine learning model to the contents of the buffer window to predict a heart rate.

27. The one or more computer memory units of claim 24, for each ultrasound image of the at least a portion of the ultrasound images of the sequence:
determining whether the single-value representation of the ultrasound image is a peak within the buffer window;
among two or more single-value representations of ultrasound images of the sequence determined to be peaks, determining an average time period between successive pairs of these single-value representations of ultrasound images of the sequence determined to be peaks; and
inverting the determined average time period to obtain a heart rate wherein the machine learning model is a direct regression model.

28. The one or more computer memory units of claim 24, for each ultrasound image of the at least a portion of the ultrasound images of the sequence:
determining whether the single-value representation of the ultrasound image is a peak within the buffer window;
among two or more single-value representations of ultrasound images of the sequence determined to be peaks, determining an average time period between successive pairs of these single-value representations of ultrasound images of the sequence determined to be peaks; and
inverting the determined average time period to obtain a heart rate, the method further comprising training the machine learning model using a plurality of observations each comprising buffer window contents for a human subject and a heart rate independently and contemporaneously determined for the human subject.

29. The one or more computer memory units of claim 24, the method further comprising causing the obtained heart rate to be displayed.

30. The one or more computer memory units of claim 24, the method further comprising causing the obtained heart rate to be stored in connection with identifying information for the person.

31. The one or more computer memory units of claim 24, the method further comprising:
for each ultrasound image of the at least a portion of the ultrasound images of the sequence:
performing filtering on the multivalued representation of the ultrasound image before the multivalued representation of the ultrasound image is accessed.

32. The one or more computer memory units of claim 24 wherein the multivalued representation of the ultrasound image is the ultrasound image itself.

33. The one or more computer memory units of claim 24 wherein the multivalued representation of the ultrasound image is a set of object detection results obtained for the ultrasound image.

34. The one or more computer memory units of claim 33, the method further comprising:
for each ultrasound image of the at least a portion of the ultrasound images of the sequence:
applying a trained machine learning model to the ultrasound image to produce the set of object detection results.

35. The one or more computer memory units of claim 33, the method further comprising:
using ultrasound images to train the applied machine learning model.

36. The one or more computer memory units of claim 24 wherein the multivalued representation of the ultrasound image is a vector of values, each value of the vector corresponding to a different ultrasound view and representing a determined probability that the ultrasound image was captured from that ultrasound view.

37. The one or more computer memory units of claim 36, the method further comprising:
for each ultrasound image of the at least a portion of the ultrasound images of the sequence:
applying a trained machine learning model to the ultrasound image to produce the vector of values.

38. The one or more computer memory units of claim 37, the method further comprising:
using ultrasound images to train the applied machine learning model.

39. The one or more computer memory units of claim 24 wherein the multivalued representation of the ultrasound image is a set of segmentation results obtained for the ultrasound image.

40. The one or more computer memory units of claim 24 wherein the multivalued representation of the ultrasound image is a set of image classification results obtained for the ultrasound image.

41. A method in a computing system, comprising:
accessing an M-mode ultrasound image representing ultrasound data received for a patient during a distinguished period of time;
for each of a plurality of vertical lines of the image corresponding to a different time during the distinguished period, each vertical line comprising a first number of values:
compressing the vertical line to transform the vertical line into a second number of values that is smaller than the first number of values;
pooling the second number of values into a single-value representation of the vertical line; and
adding the obtained single-value representation of the vertical line to a time-ordered buffer window of single-value representation representations of vertical line of the image from earlier times.

42. The method of claim 41 wherein the compression is performed by the encoder stage of an auto-encoder model trained on vertical lines of training M-mode ultrasound images.

43. The method of claim 41 wherein the compression is performed by a multi-layer perceptron.

44. The method of claim 41, further comprising:
for each of the plurality of vertical lines of the image:
determining whether the single-value representation of the vertical lines is a peak within the buffer window;
among two or more single-value representations of vertical lines of the image determined to be peaks, determining an average time period between successive pairs of these single-value representations of ultrasound images of the sequence determined to be peaks; and
inverting the determined average time period to obtain a heart rate.

45. The method of claim 41, further comprising applying a trained machine learning model to the contents of the buffer window to predict a heart rate.

46. A method in a computing system, comprising:
accessing ultrasound data collected from a person at each of a plurality of times during a distinguished period of time;
for each of the plurality of times:
compressing the ultrasound data collected from the person to obtain a single-value representation;
adding the obtained single-value representation to a buffer; and
processing the buffer contents to determine a heart rate.

47. The method of claim 46 wherein the accessed ultrasound data comprises a sequence of B-mode ultrasound images each captured at one of the plurality of times.

48. The method of claim 46 wherein the accessed ultrasound data comprises at least one M-mode image comprising vertical lines each corresponding to one of the plurality of times.

49. The method of claim 46 wherein the processing comprises performing procedural peak-finding in the buffer contents.

50. The method of claim 46 wherein the processing comprises applying a machine learning model to the buffer contents.

51. One or more computer memory units collectively having contents configured to cause a computing system to perform a method, the method comprising:
accessing an M-mode ultrasound image representing ultrasound data received for a patient during a distinguished period of time;
for each of a plurality of vertical lines of the image corresponding to a different time during the distinguished period, each vertical line comprising a first number of values:
compressing the vertical line to transform the vertical line into a second number of values that is smaller than the first number of values;
pooling the second number of values into a single-value representation of the vertical line; and
adding the obtained single-value representation of the vertical line to a time-ordered buffer window of single-value representation representations of vertical line of the image from earlier times.

52. The one or more computer memory units of claim 51 wherein the compression is performed by the encoder stage of an auto-encoder model trained on vertical lines of training M-mode ultrasound images.

53. The one or more computer memory units of claim 51 wherein the compression is performed by a multi-layer perceptron.

54. The one or more computer memory units of claim 51, the method further comprising:
for each of the plurality of vertical lines of the image:
determining whether the single-value representation of the vertical lines is a peak within the buffer window;
among two or more single-value representations of vertical lines of the image determined to be peaks, determining an average time period between successive pairs of these single-value representations of ultrasound images of the sequence determined to be peaks; and
inverting the determined average time period to obtain a heart rate.

55. The one or more computer memory units of claim 51, the method further comprising applying a trained machine learning model to the contents of the buffer window to predict a heart rate.
